(19) United States
(12) Patent Application Publication
(10) Pub. No.: US 2021/0361733 A1
(43) Pub. Date: Nov. 25, 2021

(54) Prevention and Treatment of SARS-CoV-2 Infection by Mentha piperita and Flos chrysanthemi Extracts alone, and their Combination

(71) Applicants: Atta-ur- Rahman, Karachi (PK); Muhammad Iqbal Choudhary, Karachi (PK); Saba Farooq, Karachi (PK); Atia-tul- Wahab, Karachi (PK)

(72) Inventors: Atta-ur- Rahman, Karachi (PK); Muhammad Iqbal Choudhary, Karachi (PK); Saba Farooq, Karachi (PK); Atia-tul- Wahab, Karachi (PK)

(21) Appl. No.: 17/395,491

(22) Filed: Aug. 6, 2021

(51) Int. Cl. A61K 36/534; A61K 36/287; A61P 31/14

(52) U.S. Cl. CPC A61K 36/534; A61P 31/14; A61K 36/287

(57) ABSTRACT

Due to the SARS-CoV-2 pandemic, the development and screening of novel antiviral agents are urgently required. Herein, we developed herbal extracts with promising antiviral activity against SARS-CoV-2. Antiviral effects of Mentha piperita, and Flos chrysanthemi extracts alone, in combination and their fractions were found to decrease viral load. To determine which step of the virus is inhibited by the treatment, the extracts were administered at different time points of treatment, namely prophylactic (Full-time), pre-adsorption (Entry), and post adsorption (Post-entry). Most of the fractions, exhibited high efficacy upon prophylactic administration. The viral load was lower in prophylactic-treated cells than in non-treated cells. Administration of combinations, following 2:1 molar ratio of Mentha piperita, and Flos chrysanthemi significantly reduced the viral load by increasing the Cq values. Ethyl acetate extract of Mentha piperita and hexanes extract of Flos chrysanthemi were found to be most effective at all treatment points.

### BACKGROUND OF THE INVENTION

Novel enveloped single-stranded RNA virus Severe Acute Respiratory Syndrome Coronavirus-2 (SARS-CoV-2) is responsible for causing COVID-19. SARS-CoV-2 is the seventh known coronavirus in humans, belongs to the same phylogenetic family Coronaviridae. This family includes SARS that emerged in 2002 and the Middle East Respiratory Syndrome coronavirus (MERS-CoV-2) that reported in 2012 [Andersen et al., Nat Med. 2020, 26, 450-452].

SARS-CoV-2 causes a respiratory infection with a highly variable clinical course that is dependent on pathogenesis of SARS-CoV-2 and other host factors. Mild disease, observed in 81% of patients in the initial Wuhan report, manifests self-limited respiratory symptoms typical of a viral pneumonia, including fever, cough, dyspnea, sore throat, as well as anosmia, and dysgeusia [Giacomelli et al., Clin Infect Dis. 2020, 71, 889-890]. Currently SARS-CoV-2 is global pandemic without any effective drug available for treatment. It spreads quickly and can cause death in some infected patients. The rapid emergence of COVID-19 in recent month has led to collapse of the health care systems. Rapid diagnostics and effective preventive measures are required to control it from spreading. In addition, more efforts are urgently required to develop effective and safe vaccines, and antiviral drugs [Zheng et al., Int Biol Sci. 2020, 16, 1678-1685].

As SARS-CoV-2 has spreads globally, and the virus is fast mutating, it is critical to isolate SARS-CoV-2 viruses to characterize their ability to infect and replicate in multiple human cell types, and to determine if the virus is further improving its ability to infect human cells, and cause severe diseases. So far a number of known antiviral drugs, such as lopinavir/ritonavir, darunavir/umifenovir, oseltamivir, favipiravir, remdesivir, etc. have been repurposed for the treatment of COVID-19. Other drugs, such as chloroquine, hydroxychloroquine, azithromycin, tocilizumab, interpheron-β, etc. have also been used for the treatment of COVID-19 patients.

Mentha piperita L. (Peppermint), a hybrid mint, is a cross species between watermint and spearmint, and belongs to the family Lamiaceae. Many pharmacological studies have shown that M. piperita L. possesses antioxidant, cytotoxic, antiallergenic, antiviral, and antibacterial activities with a very few side effects. The ethanolic extract from the leaves of M. piperita L. was effective against respiratory syncytial virus RSV, and could suppress the production of TNF-α, IL-6, NO, and PGE2. The anti-inflammatory activity could be beneficial in fighting RSV infections [Li et al., Food Sci Biotechnol. 2017, 26, 1675-1683]. The essential oils obtained from M. piperita were screened for antiviral activity against Herpes simplex type-1 (HSV-1), and parainfluenza type-3 (PI-3) [Orhan et al., Turk. J Biol. 2012, 36, 236-246; Schuhmacher et al. Phytomedicine. 2003, 10, 504-510]. Peppermint (M. piperita) extract also possesses antiviral activities against Newcastle disease (NDV), Herpes simplex, vaccinia, Semliki Forest, and West Nile viruses in egg and cell-culture model [Herrmann et al., SEBM. 1967, 124, 874-8].

Flos chrysanthemum was reported to have strong antiviral activity against flu virus (Han X et al., Chin J Nat Med. 2016, 14, 794-800]. The fermented beverage made of Flos chrysanthemi and Periostracum cicadae is full of nutrition, along with health-care effect of improving the immunity, strengthening and nourishing the liver, anti-aging, antivirus [Shijun et al., Faming Zhuanli Shenqing, 2018, CN 108419970 A]. Studies has shown that Flos chrysanthemi has anti-inflammatory and antibacterial activities and is effective in treating infectious diseases [Jing et al., Biomolecules. 2019, 9, 518]. Antiviral activity (esp. coronavirus, such as SARS-CoV, SARS-CoV-2) of Flos chrysanthemi in combination with traditional Chinese medicine's raw materials has also been reported [Runhua et al., Faming Zhuanli Shenqing. 2020 CN 111803593 A 20201023].

### BRIEF SUMMARY OF THE INVENTION

The current coronavirus (COVID-19) pandemic is exacerbated by the absence of effective therapeutic agents. An efficient strategy in response of this issue would be identification of potential medicinal plants with potent antiviral activity. Among many medicinal plants, Mentha piperita, and Flos chrysanthemi, were found to possess anti-viral potential against SARS-CoV-2.

The antiviral activity of ethanolic extracts of Mentha piperita, and Flos chrysanthemi was carried out after SARS-CoV-2 virus propagation, isolation, and conformation by qRT-PCR. Reduction in viral load was also evaluated by using a combination of herbal extracts in different ratios. Anti SARS-CoV-2 activity of number of fractions of plant extracts were also evaluated. Results indicated that Mentha piperita alone inhibited virus at the “full-time”, “entry” and “post entry” treatment points. However, when used in combination with Flos chrysanthemi, the inhibitory efficacy was increased in “post-entry” to some extent. Chloroquine phosphate and Remdesivir, were used as the reference drugs that have shown inhibition of SARS-CoV-2 at viral full entry and post entry treatment points (FIGS. 6 and 7), which is in accordance with other studies [Wang et al., Cell Res. 2020, 30, 269-271].

### BRIEF DESCRIPTION OF THE DRAWINGS

FIG. 1 depicts the propagation and cytopathic effects of SARS COV 2. Infection in Vero cell lines (Monkey kidney epithelial cell lines; ATCC CCL81) was observed under a light microscope at 10, 20, and 40×. A, B, and C represent the control Vero cells on different magnifications with normal morphology, size, and growth. D, E, and F are the cells after 4 days of infection with SARS-CoV-2 (4.d.p.i.(days post infection); Cell rounding, deformation, and inclusion bodies along with rough surface were observed. A large number of broken cells were also observed; up to 80% of the monolayer of Vero cell line was infected after 4 days.

FIG. 2 depicts the qRT-PCR for quantification of propagated SARS-CoV-2 in Vero cell line. Quantitative PCR of the infected Vero cell line after 4.d.p.i depicted a low Cq value, indicating a high viral titer of viral targets ORF lab, and other viral target gene. Black line with diamonds represents the ORF lab; grey plain line indicates the other viral target while plain black line is internal control of the PCR reaction.

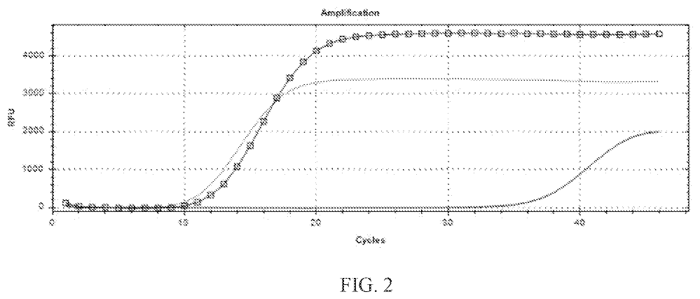

FIG. 3 depicts the cytotoxicity of herbal extracts on Vero cell line. Cytotoxicty was evaluated by MTT assay. Bar graph shows the percent inhibitions of selected herbal extracts. Chloroquine and Remdesivir was used as positive control with no inhibition. Flos chrysanthemi, and Mentha piperita were found to be non-cytotoxic, and thus selected for anti-viral screening.

FIG. 4 depicts the antiviral activity of plant extracts on different treatment points. The inhibitory efficacy of Flos chrysanthemi, and Mentha piperita by using qRT-PCR in term of Cq values was evaluated. (a). Black line with squares indicates the FC treated sample while grey line with squares indicates virus control at the “full time” treatment point, black line with circles and grey line with circles are FC treated and control samples respectively at “entry” treatment point. Lines with diamonds represent the “post entry” treatment point, in which the black line represent the FC treated sample whereas grey with diamonds indicate the virus control. Furthermore, all the plain lines in grey color represent the other viral target gene of SARS-CoV-2 while black line with crosses is the positive control and black line with triangles is the negative control (b). Black line with squares indicates the MP treated sample while grey line with squares indicates virus control at the “full time” treatment point, black line with circles and grey line with circles are MP treated and control samples respectively at “entry” treatment point. Lines with diamonds represent the “post entry” treatment point, in which the black line represent the FC treated sample whereas grey with diamonds indicate the virus control. Furthermore, all the plain lines in grey color represent the other viral target gene of SARS-CoV-2 while black line with crosses is the positive control and black line with triangles is the negative control.

FIG. 5 depicts the antiviral activity of combinations of plant extracts on different treatment points. It indicates the inhibitory efficacy of Flos chrysanthemi, and Mentha piperita by using qRT-PCR at 2:1 molar ratio. Black line with squares indicates the 2:1 combination of MP:FC treated sample while grey line with squares indicates virus control at the “full time” treatment point, black line with circles and grey line with circles are MP:FC treated and control samples respectively at “entry” treatment point. Lines with diamonds represent the “post entry” treatment point, in which the black line represent the MP:FC treated sample whereas grey with diamonds indicate the virus control. Furthermore, all the plain lines in grey color represent the other viral target gene while black line with crosses is the positive control and black line with triangles is the negative control.

FIG. 6 depicts the percent viral copy number at different treatment points. Graphs represents the percent viral copy number of crude extracts alone and in combination, along with various fractions of Flos chrysanthemi (FC), and Mentha piperita (MP) at “full time”, “entry” and “post entry” points. FC-B, FC-H, and FC-EA represents fractions of butanol, hexanes, and ethyl acetate respectively. Whereas MP-H, and MP-EA represents fractions of hexanes and ethyl acetate respectively.

FIG. 7 depicts the comparison of percent viral copy number at different treatment points. It indicates the comparison of percent viral copy number of highly effective fractions of Flos chrysanthemi (FC), and Mentha piperita (MP) at all treatment points. FC-H, and FC-EA represents hexanes and ethyl acetate fractions. Whereas MP-H and MP-EA represents fractions of hexanes and ethyl acetate.

### DETAILED DESCRIPTION OF THE INVENTION

The present invention is valuable as herbal extracts can be used as potential protection and treatment candidate against SARS-CoV-2.

Cytotoxicity Evaluation of Herbal Extracts on Vero Cell Line

Cell lines: Vero cell line (Monkey kidney epithelial cell line; ATCC CCL-81) was acquired from the Bio-Bank facility of PCMD-ICCBS. Cells were allowed to grow in tissue culture flasks T-25/T-75 in Dulbecco's Modified Eagle Medium (DMEM), supplemented with optimized percentage of Fetal Bovine Serum (FBS) and penicillin/streptomycin (Pen/Strep). The cells were incubated in a humidified atmosphere of 5% CO₂, and at 37° C. The protocol of Ammerman et al., was followed [Ammerman et al., Curr Protoc Microbiol. 2008, 11, A-4E].

Assay protocol: To evaluate the in-vitro viability of selected herbal extracts, MTT assay was performed, as previously described by Moradi et al., 2016 with slight modifications. Briefly, Vero cells were seeded in 96-well plates in growth medium (DMED, FBS, Pen/Strep) for 24 h. After incubation, the medium from the 96-well plates was aspirated and cells were washed with 1× phosphate buffered Saline (PBS). Ethanolic extracts of Flos chrysanthemi (FC), and Mentha piperita (MP), were added in different concentrations to the cultured cells in triplicate, and further incubated for 24 h post-treatment, in order to determine the cytotoxic effects. After respective period of time, the medium was removed from the plates, and the monolayer was washed with 1× PBS before addition of MTT dye (5 mg/mL). The plates were further incubated at 37° C. in 5% CO₂ for 4 h. After incubation, DMSO was added for 15 to 20 min and absorbance was measured at 540 nm with a spectrophotometer (BioRad, USA). Percentage growth inhibition was calculated by using the following formula:

$$Cytotoxic concentration ( \% ) = [ - \frac{Absorbance of test}{Absorbance of solvent control} \times 100 ]$$

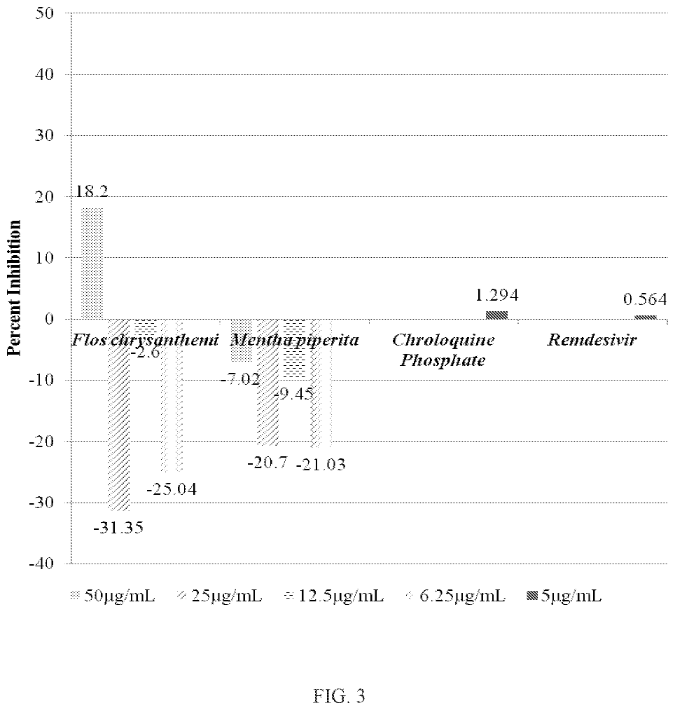

Results of cytotoxicity of medicinal plants by MTT assay: Cytotoxicity of the 7 medicinal plant extracts were carried out on Vero CCL-18 cells via MTT assay. Ethanolic extracts of Mentha piperna, and (MP), Flos chrysanthemi, were found to be noncytotoxic on Vero cell line at a dose of 50, 25, 12.5, 6.25 μg/mL. Therefore, these two were selected for anti-viral studies. In addition, various combinations of Flos chrysanthemi and Mentha piperita, in different molar ratios, were also evaluated for cytotoxicity against Vero cell line, all combinations were observed as non-toxic against Vero cell lines. Chloroquine phosphate and Remdesivir were used as a reference drugs, and found to be non-toxic. (FIG. 3).

Virus Isolation and Propagation

Specimen collection: SARS-CoV-2 isolation and propagation was performed in the BSL-3 (Biosafety Level 3) Laboratory at the International Center for Chemical and Biological Sciences (ICCBS). Virus (SARS-CoV-2) was isolated from the viral transport medium (VTM) of nasopharyngeal swabs of COVID-19 PCR positive samples, analyzed at COVID-19 testing laboratory of the ICCBS. VTM vials were stored at −80° C., and transferred to 4° C. in the BSL-3 units for propagation.

Assay protocol: The virus isolation and propagation process were initiated in T-25, and T-75 flasks with a 70 to 80% confluent monolayer of Vero cell line (Monkey kidney epithelial cell line; ATCC-CCL81). Initially, 500 μL VTM containing virus was added into the flask, with DMEM-high glucose (Thermo Fisher Scientific Inc, U. S), 2% FBS (Thermo Fisher Scientific Inc, U.S), and Penicillin-Streptomycin solution (Pan-Biotech GmbH, Aidenbach, Germany), and incubated at 37° C. for an hour with rocking every 15 mins to allow uniform virus adsorption and distribution. After the completion of incubation time, the inoculum was aspirated, cells were washed with 1X phosphate buffered saline (PBS), and flasks were again supplemented with growth medium. Each day, cytopathic effects (cellular morphological changes/growth reduction) were observed under an inverted microscope (Optika Microscope, IM-3, Italy). Virus replication and isolation with observed cytopathic effects CPE were further confirmed by taking 140 μL of supernatant and testing by qRT-PCR. Virus isolate was harvested when more than 80% of the cells manifested CPE. The culture medium was centrifuged to remove the cell debris, aliquoted, and stored at −80° C. for further passaging, and experimentation. The viral isolate was named as SARS-CoV-2-NIVPCMD-2020-1.

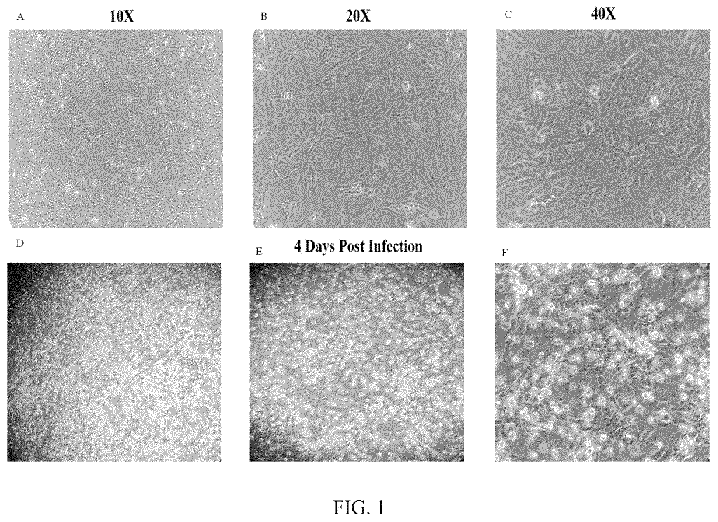

Results of viral propagation, and isolation in terms of cytopathic effects (CPEs) of SARS-COV-2 on Vero cell line: The CPEs were characteristic of SARS-CoV-2-NIVPCMD-2020-1 infection with cell rounding, detachment of the cell monolayer, and formation of loose cells on the surface. After 24 hours of infection in Vero cells, no CPEs were observed in the cell monolayer. After 36 hours, Vero cells were observed to have detectable CPE upto 40-50%, while between 48 and 72 hours of post infection (h.p.i.), the observed CPE reached approximately 90% (FIG. 1). Marked CPE were harvested at 72 h.p.i. The 72 hours SARS-CoV-2 CPE represents the phenotypic consequence of viral infection, and replication in cells. CPE was focal, with cell rounding, and a refractivity that was followed by cell detachment; and CPE quickly spread to involve the entire cell monolayer. These morphological changes are indications of the cellular infection by viruses. SARS-CoV-2-NIVPCMD-2020-1 propagation was confirmed by the presence of high levels of virus-specific RNA with the help of quantitative PCR. The virus isolate was also titrated after many passages, following the isolation (P1 to P6). In contrast, HepG2 cells did not produce a SARS-CoV-2 specific CPE during this timeframe, while the CPEs were more clearly observed in Vero CCL-81 cells. RNA quantification of every passage was monitored with low Cq value (FIG. 1).

Discussion: During the current study, the virus isolation and propagation were carried out in Vero cell lines. Marked CPEs were observed including swelling or shrinkage, rounding, lysis, plaques, clumping, syncytia, and inclusions (FIG. 1). Viral stocking and passaging were done. The confirmation of viral targets in the propagated lines was evaluated by qRT-PCR. The viral invasion, replication, and then exocytosis that causes structural, metabolic or functional alterations in the cell that is being infected (Lowenstein, C. J), (Oxford, A. E), called as cell cytopathic effect (CPE). The virus itself is not visible under light microscope; therefore, CPE is the key indicator of viral infection [Medical Microbiology, 4th edition, Chapter 44s].

Viral Titration-Median Tissue Culture Infectious Dose (TCID₅₀/mL)

Assay protocol: Vero CCL-81 cells were seeded into 96-well plate (2.5×10⁴ cells/mL), 24 h before the experiment. 96-Well plate was transferred into BSL 3 for virus titration. For 50% tissue culture infectivity dose, SARS-CoV-2 virus was diluted 1:10 in infection medium (10⁻¹ to 10⁻¹⁰). The growth medium was removed from 96-well plates; virus dilutions were applied in 8 wells, and incubated at 37° C. Visualizations were performed daily in an inverted light microscope to observe the CPE. After 72 h, the last reading was taken, and the monolayers were fixed with 10% formaldehyde buffer, and stained with 0.1% crystal violet. The wells were marked as “positive CPE” if cells had developed observable CPE in 30% or more of the surface of the wells compared to both the undiluted virus controls. Cells that did not showed such conditions were marked as “negative CPE”. The viral titer was expressed in TCID₅₀/mL, and the end-point was calculated according to Reed and Muench method based on eight replicates for titration [Reed and Munech, Am J Epidemiol. 1938, 27, 493-497].

$${log}_{10} 50 \% end point dilution = ( {log}_{10} of dilution showing a mortality next above 50 \% ) - ( difference of logarithms \times logarithm of dilution factor )$$

$$Difference of logarithms = [ \frac{( mortality at dilution next above 50 \% ) - 50 \%}{( mortality next above 50 \% ) - ( mortality next below 50 \% )} ]$$

Results of virus titration through TCID₅₀: The presence of infectious virus (SARS-CoV-2) in the cell culture supernatant was determined at regular intervals throughout the incubation period by tissue culture infectious dose 50% (TCID₅₀) assay, starting with the inoculum preparations and up to the last day of incubation. Marked cytopathic effect was recorded from the 48 h.p.i till the end of the experiment by means of an inverted optical microscope. After incubation, the median tissue culture infectious dose (TCID₅₀) was determined by the Reed and Muench method [Reed and Munech, Am J Epidemiol. 1938, 27, 493-497], based on eight replicates for titration.

$$Difference of logarithms = [ \frac{( 62.5 ) - 50}{( 62.5 ) - 12.5} ] = \frac{12.5}{50} = 0.25$$

$${log}_{10} 50 \% end point dilution = \text{-} 10 - ( 0.25 \times 1 ) = 10.25$$

$$50 \% end point dilution = {10}^{\text{-} 10.25}$$

$$Viral titer = {10}^{10.25} {LD}_{50} \text{/} mL$$

Discussion: TCID₅₀ signifies the concentration at which 50% of the cells are infected when a test tube or well plate, upon which cells have been culture, is inoculated with a diluted solution of viral fluid [Reed and Munech, Am J Epidemiol. 1938, 27, 493-497]. During the current experiments, the tissue culture infection dose was evaluated, which will be used for further investigations. This protocol also provides the end point CPE results on the dilution of 10⁻⁶.

Determination of Antiviral Potential of Herbal Plant Extracts

Assay protocol: For the “Full-time” treatment, the Vero cells CCL-81 were pre-treated with Mentha piperita, and Flos chrysanthemi extracts (alone, and in combination), fractions and drugs for 1 h, after that the virus (SARS-CoV-2) was applied for 2 h to allow infection. The extract-virus mixture was removed, and the cells were washed twice with PBS. Subsequently, the cells were incubated in the presence of fresh medium, containing plant extracts, the cellular supernatant were collected at 48 hpi for viral quantification. For the “entry” condition, the cells were treated with the standard drugs, plant extracts (alone and in combination) and fractions of plant extracts for the infection period (2 h), followed by removal of the drug-virus/extract-virus/fraction-virus mixture, and washing of the cells. Subsequently, the cells were incubated in infection medium without the drugs for 48 h. For therapeutic condition, following viral infection without the test samples (standard drugs, plant extracts and fractions of plant extracts) for 2 h, the virus was discarded, and the cells were washed. Then, plant extract in fresh infection medium were added to the cells for 48 h. Results were investigated by qRT-PCR. The protocols of Cho and Wang [Cho et al., Sci Rep. 2020, 10, 16200; Wang et al., Cell Res. 2020 30, 269-271 were adapted with minor modifications.

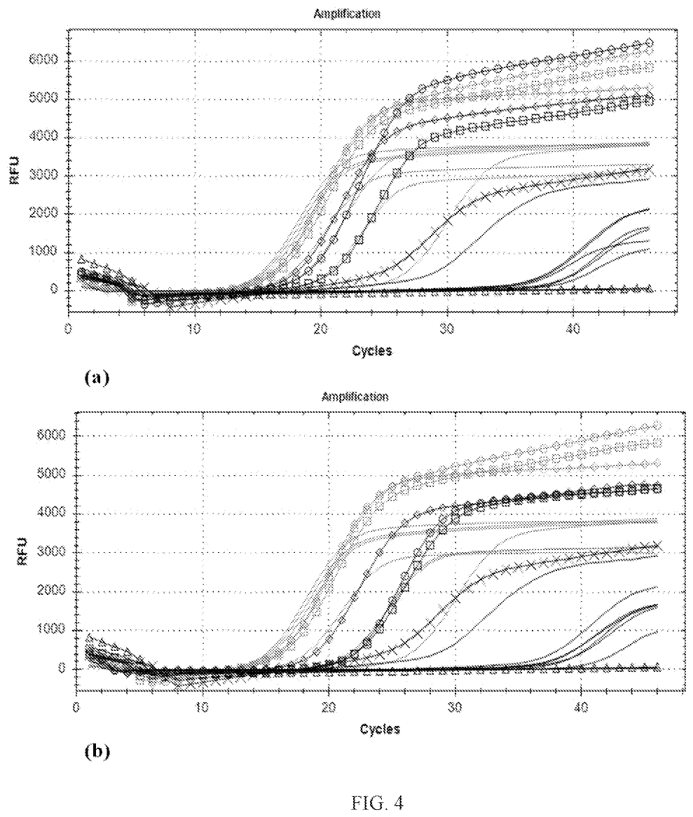

Results of anti-viral potential of medicinal plants: Extracts of Mentha piperita, and Flos chrysanthemi, were found to have anti-SARS-CoV-2 activities. To understand this impact more extensively, the interference of these plants extracts and their fractions (alone, and in combinations) were evaluated on virus “full-time”, “entry,” and “post-entry” treatment points. The antiviral effect was evaluated by quantifying the Cq values, observed in the supernatant of the SARS-CoV-2 infected Vero cell line via qRT-PCR. Chloroquine phosphate (9.69 μM/mL) and Remdesivir (8.297 μM/mL) were used as the reference drugs that exerts the viral load inhibition by increasing the Cq value, and decreasing the percent viral copy number. Results of qRT-PCR demonstrated that Mentha piperita (MP), was effective against SARS-CoV-2 propagation in Vero cell lines by increasing the Cq values in full-time (FIG. 4a) indicating the reduction in viral load.

Flos chrysanthemi (FC), exhibited more pronounced anti-SAS-CoV-2 activity at “entry” and “post entry “treatment points (FIG. 4b). However, the extract of Mentha piperita (MP) was found to be the most effective at “full time” treatment point. It also reduced the viral copy number in “entry” and “post entry” treatment points.

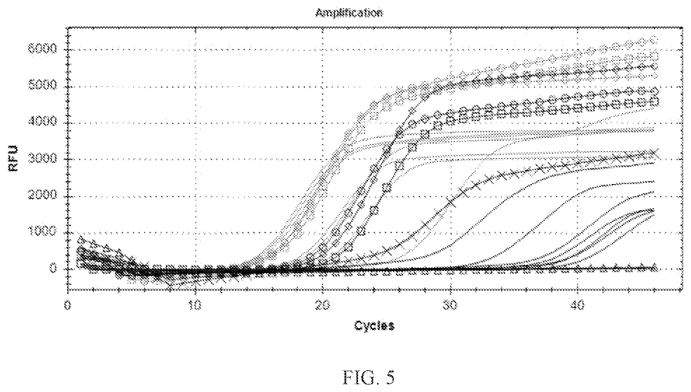

Various combinations of the herbal extracts (MP, and FC) were also evaluated on the replication of SARS-CoV-2 in Vero cell lines. Among them the combination of MP: FC (2:1; 50 ug/mL) was found to be the most effective one, which inhibited the viral load (SARS-CoV-2) over 96% in both “full time” and “post-entry” treatment points. This is an indication of prophylactic, and therapeutic potentials of the MP: FC (FIG. 5).

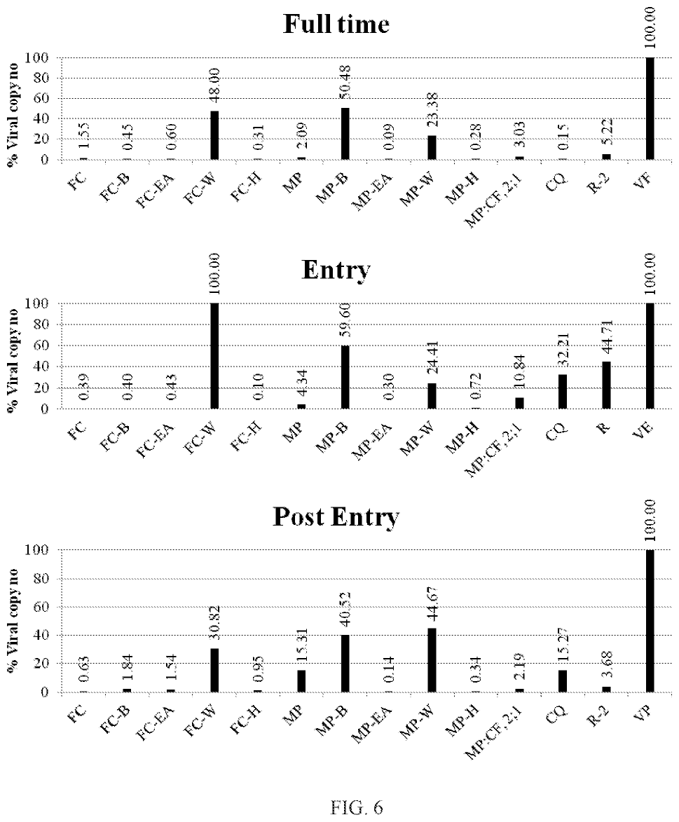

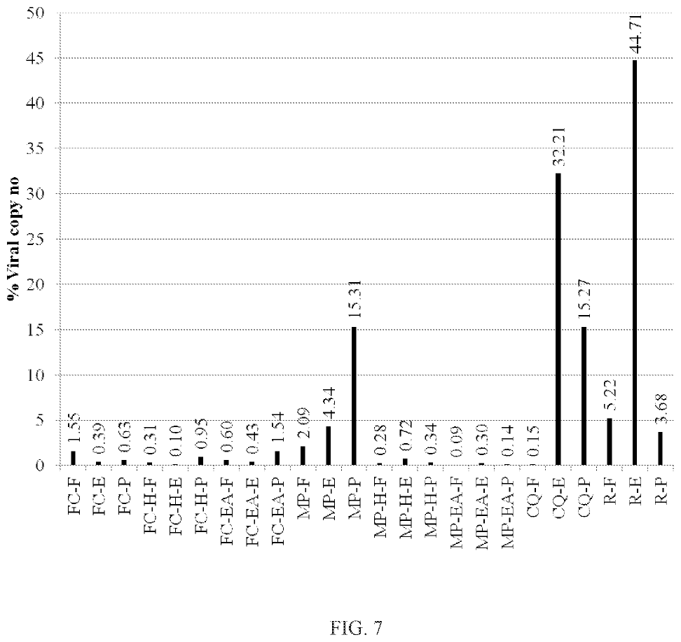

Subsequently, screening of fractions of plant extracts was also carried out in order to search for the new and promising anti-SARS-CoV-2 candidates. Hexanes and ethyl acetate extracts of FC exerts most pronounced effect with higher Cq values at all the treatment points when compared with the Cq of virus controls. Butanol extract of FC lowers the viral load in a similar pattern in both “full time and entry” points. This finding encourages further purification of compounds to identify the potent antiviral compounds within these extracts. Similarly, the hexanes and ethyl acetate fractions of MP have shown major inhibitory effect on all the treatment points (FIGS. 6 and 7). These results were also confirmed by calculating the fold change from the Cq values of the treated samples, as compared to the control by using the ΔCq method (fold changed in viral RNA=2\hat{ }ΔCt), and expressed as % of virus alone.

Discussion: In a study, hydroxychloroquine was reported with significant antiviral activity in the prophylactic/therapeutic (full-time) experimental setting both at 37° and 4° C., evident both by CPE and RT-PCR analyses [Clementi et al., Front. Microbiol. 2020, 11, Article 1704]. Chloroquine is known to block virus infection [Wang et al., Cell Res., 2020, 30, 269-271].

Plants derived compounds are promising agents for finding the viral entry inhibitors as drug candidate. Coronaviruses enter the cell after binding to the host cell receptor, angiotensin-converting enzyme 2 (ACE2). The entry of virus is achieved via attachment of SARS-CoV spike (S) protein to receptor ACE2. Many different phytochemicals have shown a strong inhibition of the interaction of SARS-CoV S protein and ACE2. Such as anthraquinone compounds; emodin, and rhein, and a flavonoid; chrysin, derived from genus Rheum and Polygonum were tested for their activity against SARS-CoV [Jahan and Onay, Turk J Biol. 2020, 44, 228-241]. During the current study, the tested herbal extracts (MP, and FC; 50 μg/mL) exhibited their effect on “full-time” as well as “entry” treatment points while in combination (2:1, 50 μg/mL), also produced some level of viral inhibition at post-entry level. Both fractions also exerted anti-SARS-CoV-2 inhibition at different treatment points by decreasing the viral load with the increased Cq values.

Butanol extract of FC was found to be effective and suggested that highly polar compounds were responsible for producing activity against SARS-CoV-2.

What is claimed is:

1. A method of protection, and treatment of COVID-19 through the administration of an effective dose of herbal extracts of Mentha piperita and Flos chrysanthemi alone, and in combination, in suitable pharmaceutical excipients, adjuvant, carrier, or diluent to humans and animals in need thereof.
